(19) United States
(12) Patent Application Publication
(10) Pub. No.: US 2003/0075186 A1
(43) Pub. Date: Apr. 24, 2003

(54) Oral appliance and method for use in weight loss and control

(76) Inventor: Michael J. Florman, New York, NY (US)

Correspondence Address: FAY, SHARPE, FAGAN, MINNICH & McKEE, LLP, Seventh Floor, 1100 Superior Avenue, Cleveland, OH, 44114-2518, US

(21) Appl. No.: 10/262,038

(22) Filed: Oct. 1, 2002

Related U.S. Application Data
(60) Provisional application No. 60/326,417, filed on Oct. 1, 2001.

(51) Int. Cl. A61C 7/16; A61C 7/20; A61B 17/56

(52) U.S. Cl. 128/869; 128/859; 433/19; 433/9

(57) ABSTRACT

An oral appliance device and method are provided for slowing the ingestion of food thereby triggering an increase in the feeling of satiety for a given amount of food. The appliance affixes to the maxillary teeth and mandibular teeth, and restricts the distance the wearer can open the mouth. The appliance includes upper and lower attachable portions which affix to the upper and lower teeth, and a flexible element that connects the upper and lower parts. The restricted opening distance can be adjusted, by either lengthening the flexible part of the appliance, or re-positioning the upper and lower affixable part(s). The appliance allows the patient to perform oral hygiene and speak normally. The appliance allows patients to chew food, but at a slower rate. The appliance decreases the amount of food a wearer can put into his/her mouth at any single time, increasing the amount of time it takes to eat.

This application claims the benefit of U.S. Provisional Patent Application No. 60/326,417 filed Oct. 1, 2001.

### BACKGROUND OF THE INVENTION

1. Field of the Invention

The invention is related to the art of weight control devices and more specifically, dental appliances operative to assist a patient in reducing the intake of food. The invention restricts mandibular movement and therefore slows the rate of food ingestion. The device also decreases the amount of food in the mouth at any given time, due to the constriction of opening, resulting in a decrease in volume. Slowing the rate of food ingestion allows the body time to respond to the ingestion of food with a sensation of satiety. The patient feels full after consuming a reduced amount of food. Therefore, the patient eats less and loses weight.

2. Description of Related Art

A number of dental appliances have been described as aiding a patient in achieving weight loss through the restriction of mandibular movements. For example, U.S. Pat. No. 6,138,679 to Renders, et al. discloses a mandibular restraint that includes a pain-inducing device. The pain-inducing device can be a bar shaped element with a thickened portion that is said to press against a gum or jaw of the patient as the patient attempts to open the mouth beyond a threshold position. Alternatively, or additionally, the pain-inducing device delivers a painful electric shock to the gum or tooth of the patient.

Russian Patent SU 1602507 A1 shows interlocking rings attached to opposing upper and lower teeth. The rings appear to limit mandibular movement.

These prior art devices have several disadvantages. For example, patients object to being shocked or painfully poked. Additionally, rubbing, poking or pressing the gums as described above can lead to abrasions, cuts and infection. Furthermore, in order to reduce a risk that mandibular movements may dislodge the teeth that the movement limiting apparatus are attached to, more than one apparatus should be installed. For example, two or four sets of teeth should be involved in limiting the opening of the jaw. Involving additional sets of teeth requires that the restraints be synchronized, or set to apply equal amounts of restraining tension, so that the teeth share the load of jaw restraint evenly. The prior art does not include a method or apparatus for restraint synchronization.

### BRIEF SUMMARY OF THE INVENTION

For the forgoing reasons, a system and method for helping a patient lose weight has been developed. The system includes restraining elements or fibers operative to be attached to, mounted to or associated with upper and lower teeth. The restraining elements or fibers prevent the mouth from opening beyond a predetermined restraint distance. The method includes procedures for synchronizing a plurality of restraining elements. Synchronizing the elements allows the elements and the teeth that they are attached to, or associated with, to share the load of jaw restraint equally, thereby minimizing a risk of tooth movement. Optionally the system includes brackets that assist in the synchronization procedure. In one embodiment the bracket includes a plastic pawl. The pawl releasably catches on notches in a restraining fiber tab. In another embodiment the bracket includes a buckle clasp type member for frictionally holding a restraining fiber tab. Another embodiment includes a locking button bracket. The button includes frictional grooves for intertwining and locking a restraining fiber. In yet another embodiment a bracket and bracket cap include interlocking convolutions. A restraining fiber is frictionally captured within the convolutions. Still other embodiments include a plurality of holes punched, molded or formed into tabs of restraining fibers or elements. Associated brackets include pins or bolts for lockable insertion through a selected one of the holes. One embodiment includes a gear or wheel for frictionally capturing and positioning a restraining fiber. Another embodiment includes a bracket with a slotted bolt. A fiber is threaded through the slot and secured with a locking nut.

### BRIEF DESCRIPTION OF THE SEVERAL VIEWS OF THE DRAWINGS

The invention may take form in various components and arrangements of components, and in various procedures and arrangements of procedures. The drawings are only for purposes of illustrating preferred embodiments, they are not to scale, and are not to be construed as limiting the invention.

FIG. 1 is a front view of restraining element bonded to upper and lower molars of a patient's mouth. The mouth is closed.

FIG. 2 is a side view of a bonded restraining element of FIG. 1.

FIG. 3 is a front view of restraining element bonded to upper and lower molars of a patient's mouth. The mouth is open a maximum or restrained distance.

FIG. 4 is a top and front view of a stationary arch installed on a lingual side of a set of teeth.

FIG. 5 is a side view of a restraining element bonded to upper and lower teeth in conjunction with magnets. The magnets are also bonded to the upper and lower teeth.

FIG. 6 is a front view of restraining elements attached to brackets. The brackets are bonded to upper and lower molars of a patient's mouth. The mouth is closed.

FIG. 7 is a side view of a mounted restraining element of FIG. 6.

FIG. 8 is a front view of restraining elements attached to brackets. The brackets are bonded to upper and lower molars of a patient's mouth. The mouth is open a maximum or restrained distance.

FIG. 9 is a side view of a restraining element bonded to upper and lower magnets. The magnets are, in turn, bonded to the upper and lower teeth.

FIG. 10 is a front view of an ordinary orthodontic bracket.

FIG. 11 is a set of views of a gear or wheel bracket and an associated restraining fiber.

FIG. 12 is a set of views of a cable tie style bracket and an associated restraining fiber.

FIG. 13 is a set of views of a buckle clasp bracket and an associated restraining fiber.

FIG. 14 is a set of views of a pin clasp bracket and an associated restraining fiber.

FIG. 15 is a front view of a nut and bolt bracket and an associated restraining fiber.

FIG. 16 is a set of side views of a capped bracket and an associated restraining fiber.

FIG. 17 is a set of views of a locking button bracket and an associated restraining fiber.

FIG. 18 is a set of views of a slotted nut and bolt bracket and an associated restraining fiber.

FIG. 19 is a view of a first semi permanent restraining system.

FIG. 20 is a view of a first semi permanent restraining system.

FIG. 21 is a view of a first semi permanent restraining system.

FIG. 22 Is a view of a temporary restraining system.

FIG. 23 is a flow chart outlining a method operative to help a patient lose weight.

### DETAILED DESCRIPTION OF THE INVENTION

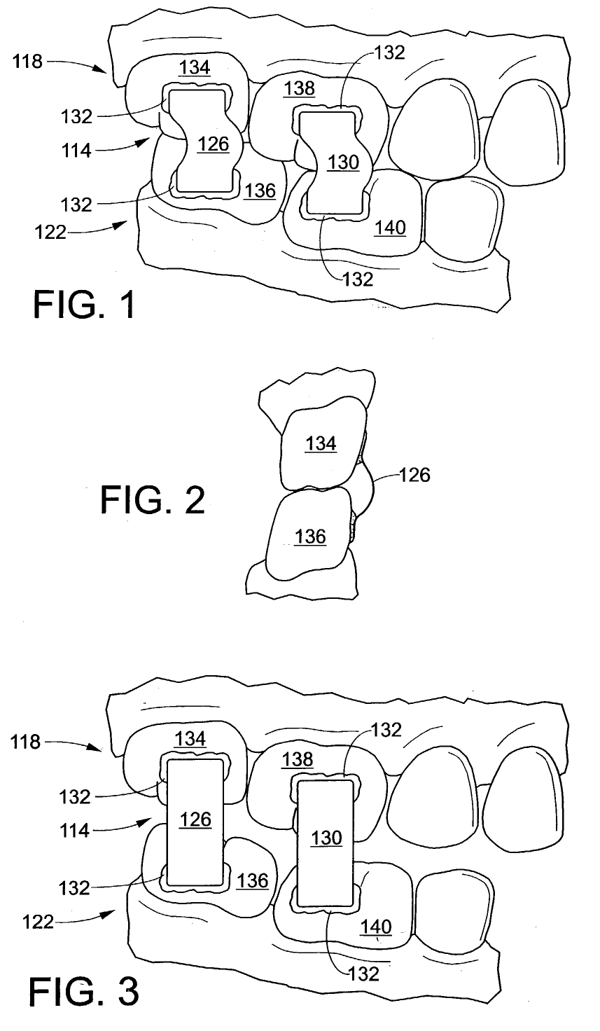

Referring to FIG. 1 and FIG. 2, which are front and side views respectively, a restraining device 114 is installed between portions of an upper jaw 118 and a lower jaw 122 of a weight loss patient (not shown). In the illustrated embodiment the restraining device includes first 126 and second 130 strips. A means for attaching the strips 126, 130 to respective portions of the maxilla or upper jaw 118 and mandible or lower jaw 122 of the patient, includes an adhesive 132. In the illustrated embodiment the respective portions of the maxilla or upper jaw 118 and mandible or lower jaw 122 are selected teeth 134, 136, 138, 140.

The first and second strips 126, 130 are made of any appropriate dental or orthodontic material. Preferably the strips are cut lengths of ultra high strength plastic mesh reinforcing material, such at that disclosed in U.S. Pat. No. 5,829,979 to Kobashigawa et al. or that disclosed in U.S. Pat. No. 5,176,951 to Rudo. These materials are very strong, while at the same time being relatively comfortable for the patient to wear. For example, these materials do not include sharp edges, and have no offensive taste. Alternatively, the strips 126, 130 are cut or stamped pieces of plastic or metal sheet or ribbon. In some embodiments, the strips 126, 130 are made of material such as, memory metal, having, for example, a preformed bow toward the cheek side of the mouth. Such a buccal bow helps prevent the strips from folding toward the teeth and being bitten when, for example, the patient chews food. As will be described in greater detail below, in some embodiments the strips are made of molded or stamped plastic, that coil or bend out of the way of chewing teeth.

As mentioned above, the strips are bonded to the teeth with an adhesive 132 such as, a dental or orthodontic resin. For example, ends 148 of the strips 126, 130 are dipped in or brushed with a low viscosity thermosetting resin such as that manufactured by Kerr Corporation under its trademark Porceline® or any orthodontic bracket or band cement, or self-curing, light or time activated, bis-GMA resin, similar to 3M Unitek Transbond (R) or other cements, similar to a glass ionomer cement, etc. is used to bond the brackets 632 to the selected teeth 634, 636, 638, 640. The saturated or coated tips are then placed against respective ones of the selected teeth 134, 136, 138, 140 and the adhesive is cured. For example, the adhesive 148 is exposed to an ultraviolet light, which causes the adhesive to harden. Of course, central portions of the strips are to remain flexible and unbonded. Therefore, adhesive is not used on the central portions of the strips 126, 130.

While restraints are shown attached to two teeth, any appropriate number of teeth can be enlisted in to the restraint. Preferably, the restraint is symmetric within the mouth. For example, where a set of left molars is selected, a set of left right molars is also selected.

The selected teeth 134, 136, 138, 140 are determined based on the oral health of the patient. Preferably the selected teeth are upper and lower first and second molars. Molars are preferably selected because molars provide a relatively large surface area for bonding and have an extensive root system. It is preferable to select teeth that are firmly held within the jaw because significant withdrawing or uprooting forces are applied to the selected teeth when, as shown in FIG. 3, the patient opens his or her mouth. This is especially true just after installation, before the patient has developed a significant muscle memory or learned the new limits of mouth opening. Additionally, unconscious mouth movements, made, for example, while the patient sleeps, can apply tooth-loosening forces to the selected teeth.

Where healthy molars are not available, other teeth may be used. For example narrower strips may be applied to non-molars and additional teeth can be selected and employed.

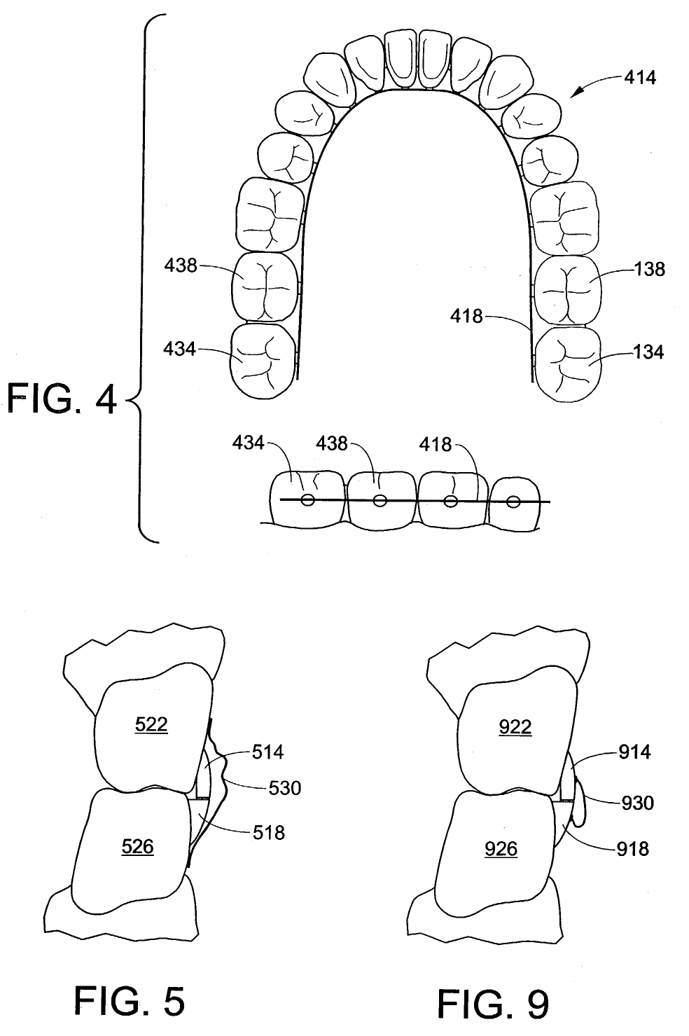

Referring to FIG. 4, stationary arches are installed in the mouth of the patient in order to combat, or compensate for these tooth loosening forces. For example, a casting is made of the maxillary 414 and mandibular (not shown) arches of the patient. The castings are used as a measure of the shape and locations of the teeth of the patient. The castings are used to form upper and lower stationary arches. The upper stationary arch is installed to support the selected teeth 134, 138 of the upper jaw. For example, an upper stationary arch 418 formed from orthodontic wire, is bonded to the lingual side of teeth that make up the maxillary arch 414, including the upper selected teeth 134, 138 shown in FIG. 1 and FIG. 2 as well as other selected upper teeth 434, 438 on the other side of the mouth of the patient. The upper stationary arch is bonded to the teeth with a bonding agent 422 in a manner known in the art. A similar arch, a lower stationary arch (not shown), is similarly installed and attached to teeth of the mandibular arch.

Alternatively, abbreviated stationary arches or arch segments are used. For example, it may not be necessary or possible to call on the front teeth for support of the selected teeth. In that case a stationary segment is bonded to the selected teeth and a few neighboring or available teeth. For example, where the selected teeth are molars, a stationary segment may be only bonded to, for example, the molars, wisdom teeth and premolars.

The restraining device is operative to control the size of bites of food taken by the patient. In addition, the restraining device hampers the eating process by making it more difficult to chew. For example, the strips 126, 130 make it difficult to transfer a bolus of food to the buccal or cheek side of the teeth. Additionally the strips make it difficult to get large pieces of food between the teeth for chewing and grinding purposes. Making eating more difficult in this way slows the rate at which food is ingested. As is known in the art of weight control, the sensation of satiety is somewhat delayed. Given enough time, a small volume of food will provide the sensation of satiety. However, many weight loss patients eat quickly. Such patients do not allow enough time for the nervous system to register the ingestion of the small volume of food, before eating an additional volume of food. Therefore, these patients eat more food than would otherwise be required to satisfy their appetites.

While the restraining device described thus far serves to reduce bite size, and thus tends to slow the eating process, some patients may learn to compensate for reduced bite size by increasing bite frequency. For such patients, a more elaborate restraining device is required.

Referring to FIG. 5, an upper magnet 514 and a lower magnet 518 are attached to respective upper 522 and lower 526 selected teeth. As illustrated, the magnets are located on the buccal side of the selected teeth. However, other mounting arrangements are possible. For example, the magnets may be located on the lingual side of the teeth. The magnets 514, 518 are oriented so that an attractive force exists between them. That is, a north pole of one magnet faces a south pole of the other magnet. With this arrangement, each attempt to open the mouth, made by the patient is initially resisted. The patient must overcome this resistance with each chew. Therefore, the chewing process is slowed.

The magnets may be coated or jacketed in order to provide a minimum gap between pole surfaces. The inclusion of such a gap producing covering is operative to attenuate the magnetic force between the magnets. Other attenuation schemes are based on magnetic material selection and magnet shape and size design.

As shown in FIG. 5 the magnets 514, 516 are bonded to the teeth 522, 526 with an adhesive (not shown). Additionally, a restraint strip 530 is separately bonded to the same teeth 522, 526. However, other arrangements are contemplated and within the scope of the invention. For example, restraint strips and magnets may be bonded to different selected teeth. Orthodontic bands or brackets may be used to mount the magnets and /or the strips. The strips may be bonded or otherwise attached to the magnets instead of directly to the teeth.

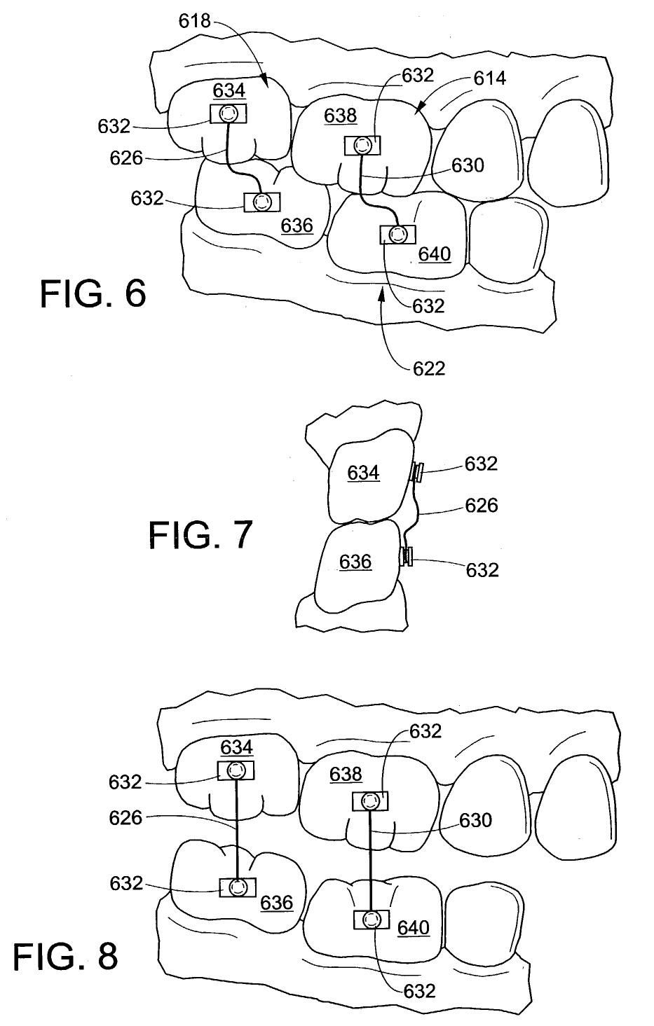

Another alternate embodiment is depicted in FIG. 6-FIG. 8. In this embodiment a restraining device 614 is also installed between portions of an upper jaw 618 and a lower jaw 622 of a weight loss patient (not shown). The restraining device 614 includes first 626 and second 630 filaments or wires. A means for attaching the filaments to portions of the upper and lower jaw includes brackets 632. The brackets are bonded to selected teeth 634, 636, 638, 640 of the maxilla or upper jaw 618 and mandible or lower jaw 622 of the patient.

The first and second filaments or wires 626, 630 are made of any appropriate dental or orthodontic material. Preferably the filaments or wires are cut lengths of orthodontic wire or plastic. In some embodiments, the wires 626, 630 are made of material such as, memory metal, having, for example, a preformed bow toward the cheek side of the mouth. As mentioned above, such a buccal bow helps prevent the filaments from folding toward the teeth and being bitten when, for example, the patient chews food.

The brackets 632 are bonded to the teeth with an adhesive 644 such as, a dental or orthodontic resin. For example, a low viscosity thermosetting resin such as that manufactured by Kerr Corporation under its trademark Porceline® or any orthodontic bracket or band cement, or self-curing, light or time activated, bis-GMA resin, similar to 3M Unitek Transbond (R) or other cements, similar to a glass ionomer cement, etc. is used to bond the brackets 632 to the selected teeth 634, 636, 638, 640. Alternatively, the brackets are attached to portions of the upper and lower jaw by some other means. For example, orthodontic bands can be used to attach brackets to teeth. Arch banding devices, such as depicted in U.S. Pat. No. 6,086,365 to Fields can be used to provide tie point for the filaments or wires 626, 630. Alternatively, interdental brackets such as those disclosed in U.S. Pat. No. 4,968,248 to McColgan et al. can be used to provide tie points at the spaces between teeth. These attachment means are exemplary only. As will be described below, other bracketing and attachment schemes can be used and stay within the scope of the invention.

Referring to FIG. 7, in the illustrated embodiment the filaments or wires 626 are looped around and tied to the brackets 632. However other attachment methods can be used and stay within the scope of the invention.

Again, the selected teeth 634, 636, 638, 640 are determined based on the oral health of the patient. Preferably the selected teeth are upper and lower first and second molars. Molars are preferable because of the extent of the molar root system. It is preferable to select teeth that are firmly held within the jaw because significant withdrawing or uprooting forces are applied to the selected teeth when, as shown in FIG. 8, the patient opens his or her mouth. Where healthy molars are not available, other teeth may be used. For example brackets may be applied to or between non-molars and more teeth can be selected and used to spread the load.

Again, where necessary, upper and lower stationary arches or arch segments are installed as described in reference to FIG. 4.

Referring to FIG. 9, in order to further slow the chewing process an upper magnet 914 and a lower magnet 918 are attached to respective upper 922 and lower 926 selected teeth. As illustrated, the magnets are located on the buccal side of the selected teeth. However, other mounting arrangements are possible. For example the magnets may be located on the lingual side of the teeth. The magnets 914, 918 are oriented so that an attractive force exists between them. That is, a north pole of one magnet faces a south pole of the other magnet. With this arrangement, each attempt to open the mouth, made by the patient, is initially resisted. The patient must overcome this resistance with each chew. Therefore, the chewing process is slowed.

The magnets may be coated or jacketed in order to provide a minimum gap between pole surfaces. The inclusion of such a gap producing covering is operative to attenuate the magnetic force between the magnets. Other attenuation schemes are possible. Some other attenuation schemes are based on magnetic material selection and magnet shape and size design.

As shown in FIG. 9 the magnets 914, 916 are bonded to the teeth 922, 926 with an adhesive (not shown). Additionally, a restraint filament 930 is attached to the magnets. In some embodiments the filament 930 is adhesively bonded to the magnets. In other embodiments the magnets or magnet jackets (not shown) are formed to include a filament attachment member, such as, for example, a tie point. Additionally, other arrangements are contemplated and within the scope of the invention. For example, restraint filaments and magnets may be bonded to different selected teeth. Orthodontic bands or brackets may be used to mount the magnets and /or the filaments. The filaments may be bonded or otherwise attached directly to the teeth instead of to the magnets.

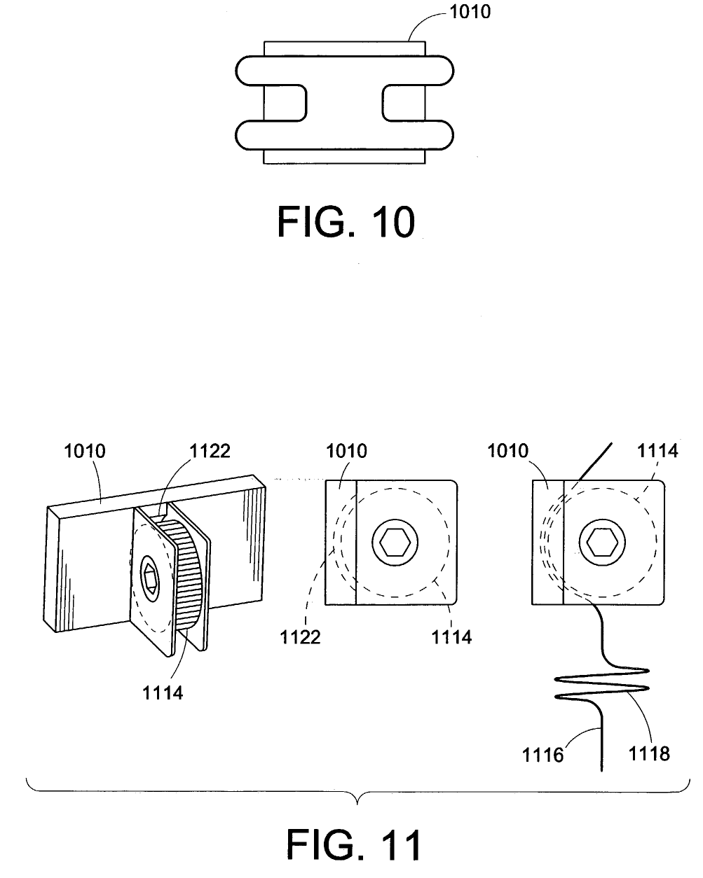

Referring to FIG. 10, any orthodontic bracket 1010 can be used to obtain sufficient retention of restraining filaments, elements, or strips. The size of orthodontic brackets useful in restraining mandibular movement can vary. Typically, useful brackets are between 5 mm and 3 mm. This dimension is dictated by the surface of the selected tooth. Preferably, brackets are not so large as to interfere with normal jaw function, nor so small that the bonding area does not allow for sufficient retention or bonding force.

The brackets should include a pad or rear surface operative to bond to a tooth or tooth side. The bonding surface of the pad is rough or mesh like so that it allows a bonding material or adhesive to affix to the pad, thereby helping the adhesive bond the bracket to the tooth. Brackets can be made from any metal or suitably strong plastic, compatible with the fluids of the oral cavity, and that are not toxic to the body.

While ordinary orthodontic brackets can be used as part of a jaw restraint, as with direct bonding of restraining filaments, the use of ordinary orthodontic brackets does not facilitate the precise adjustment of the fiber to accommodate precision fixation or synchronization. Precision fixation is the synchronized activation or engagement of a plurality of restraining elements, during jaw opening.

Preferably, the components of the jaw restraint make adjustment and restraint synchronization easy.

Referring to FIG. 11, a gear bracket 1110 incorporates a small wheel/allan wrench type key system. A fiber 1116 is placed into a curved slot 1122 behind a wheel or gear 1114. The fiber 1116 is frictionally engaged between the wheel 1114 and a wall of the groove. Alternatively, the fiber 1116 includes slots or notches for engaging teeth of the gear 1114. In either case, turning the wheel or gear 1114 (for example, with an allan wrench) advances the fiber 1116 into or out of the gear bracket, thereby providing for precision fixation. Optionally, the fiber 1116 includes a molded or cast coil portion 1118. The molded or cast coil takes up slack in the fiber when the jaw (not shown) is in a closed position.

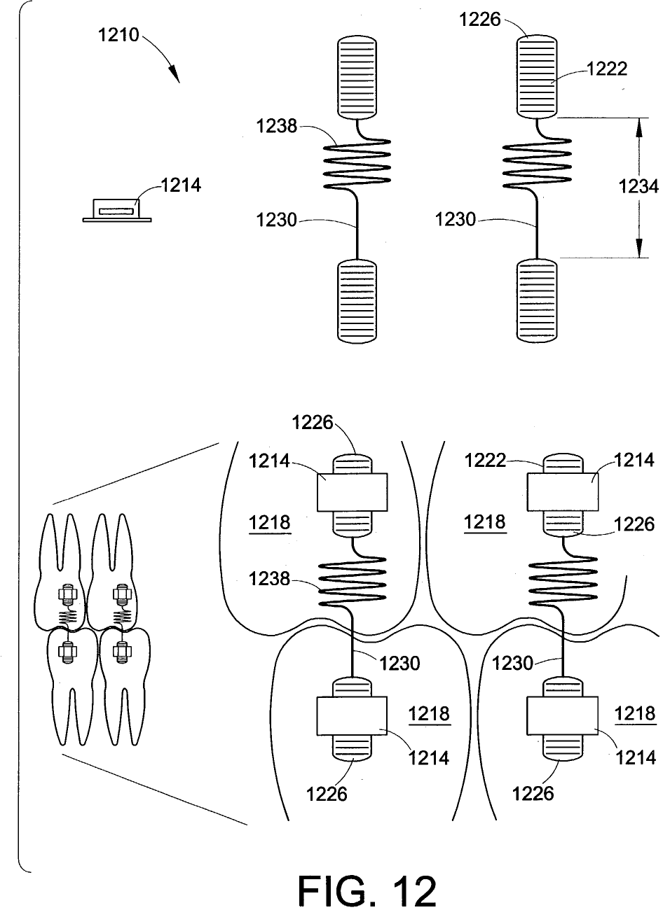

Referring to FIG. 12 a cable tie clasp bracketing assembly 1210 incorporates a female 1214 “cable tie” element for bonding to a tooth 1218. A male “cable tie” end included on a fiber can then be fit into the female 1214 element and be adjusted. For example, the female element 1214 includes a pawl (not shown) that falls into and catches on notches 1222 on a male portion 1226 of a restraining fiber 1230. Optionally, the female element includes a release mechanism that moves the pawl and allows the male portion 1226 to be withdrawn. Each fiber 1230 includes two male portions 1226. Preferably a fiber distance 1234 between the two male portions 1226 is about five millimeters. Optionally, the fiber 1230 includes a molded or cast coil portion 1238. The molded or cast coil takes up slack in the fiber when the jaw (not shown) is in a closed position.

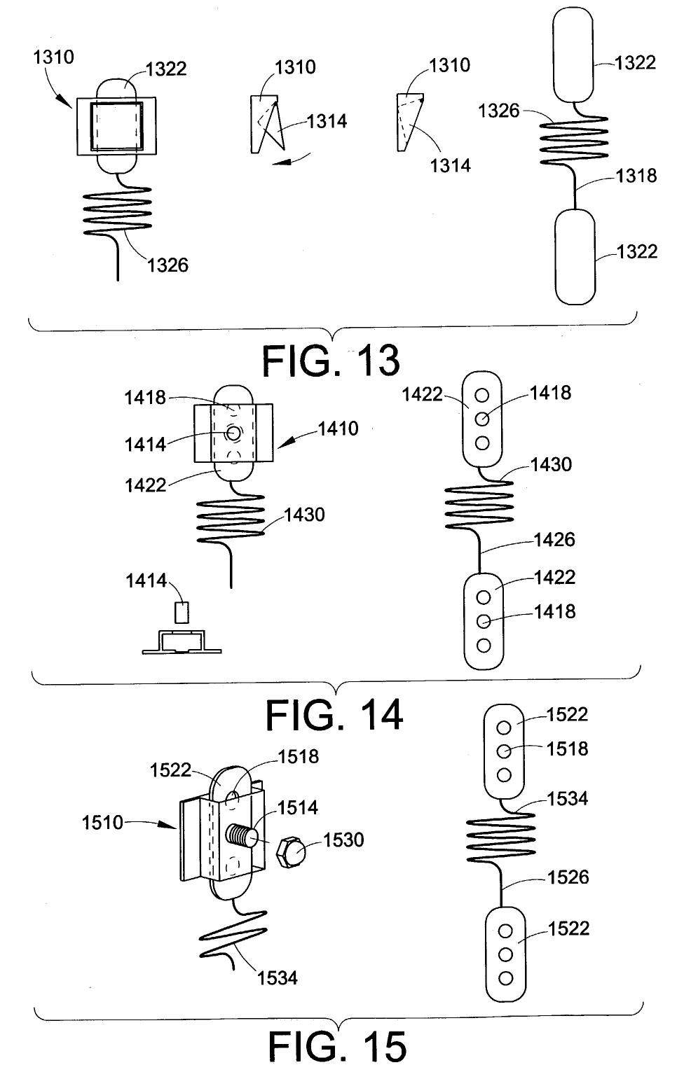

Referring to FIG. 13, a buckle clasp bracket 1310 incorporates pivoting pressure element 1314 that when engaged, tightens down on a fiber 1318. Releasing the pressure element allows the fiber to be easily adjusted in the mouth. Engaging the element frictionally traps the fiber 1318 in a desired position. Optionally, the fiber 1318 includes pressure pads or tabs 1322 for engaging the pressure element 1314. Also optionally, the fiber 1318 includes a molded or cast coil portion 1326. The molded or cast coil 1326 takes up slack in the fiber 1318 when the jaw is in a closed position.

Referring to FIG. 14 a pin clasp bracket 1410 incorporates a pin 1414 that when engaged, fits directly into small holes 1418 pre-punched, molded or formed in tabs 1422 of a fiber 1426. Removing the pin 1414 allows the position of the tabs 1422 and fiber 1426 to be easily adjusted. Installing the pin 1414 in one of the holes 1418 captures the fiber 1426 into a fixed position. Optionally, the fiber 1426 includes a molded or cast coil portion 1430. The molded or cast coil 1430 takes up slack in the fiber 1426 when the jaw (not shown) is in a closed position.

Referring to FIG. 15, a nut and bolt bracket 1510 is similar to the pin clasp bracket 1410. The nut and bolt bracket 1510 incorporates a threaded bolt 1514 that projects outward. The bolt is placed through one of a plurality or pre-punched or molded holes 1518 in a tab 1522 of a restraining fiber 1526. A sealing nut 1530 is placed over the bolt 1514, to hold the fiber 1526 in place and prevent the bolt 1514 from harming a cheek of a patient (not shown). Optionally, the fiber 1526 includes a molded or cast coil portion 1534. The molded or cast coil 1534 takes up slack in the fiber 1526 when the jaw (not shown) is in a closed position.

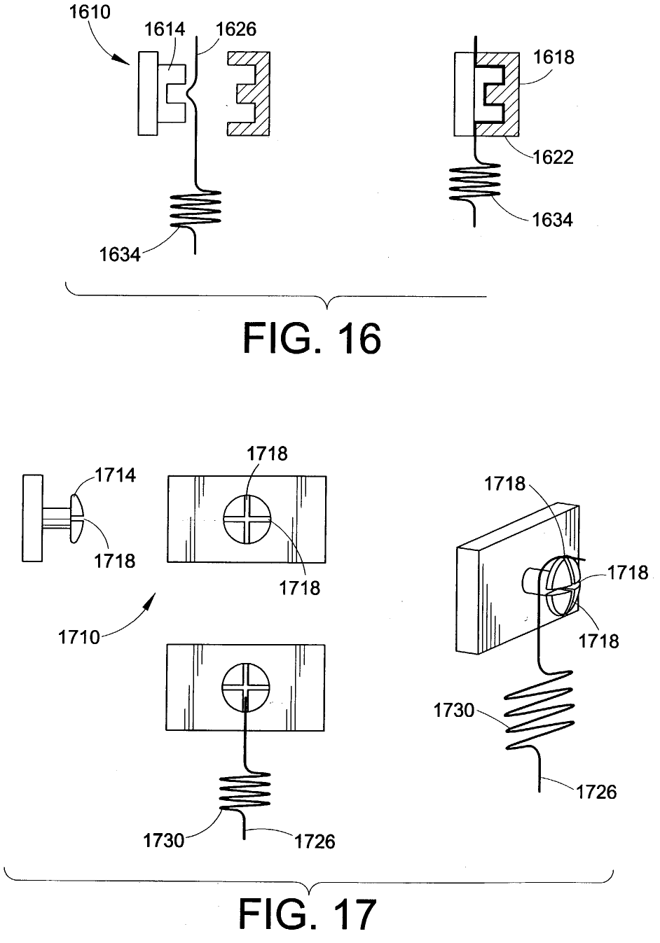

Referring to FIG. 16, a capped bracket 1610 includes a first convoluted portion 1614. A frictionally engaging bracket cap 1618 includes a second convoluted portion 1622. When the cap 1618 is installed on the capped bracket 1610, the first convoluted portion 1614 mates in an interlocking manner with the second convoluted portion 1622. In use, before the cap 1618 is placed on the bracket 1610 a fiber 1626 is properly positioned and placed between the cap 1618 and bracket 1610. The act of placing the cap 1618 onto the bracket 1610 forces the fiber 1626 to bend and conform to the shape of the mating convolutions. Wedged between the first 1614 and second convolutions 1622 the fiber 1626 is frictionally captured in position between the cap 1618 and the bracket. Optionally, the fiber 1626 includes a molded or cast coil portion 1634. The molded or cast coil 1634 takes up slack in the fiber 1626 when the jaw (not shown) is in a closed position.

Referring to FIG. 17, a locking button bracket 1710 includes a button element 1714. The button element includes a plurality of threading grooves 1718. In use, a restraining filament 1726 is properly positioned and wrapped once or twice around a shaft 1722 attaching the button to the bracket 1710. A free end of the filament 1726 is then wedged into one or more of the threading grooves 1718. The threading grooves frictionally capture the fiber. Popping the filament 1726 back out of the threading grooves 1718 allows the position of the fiber to be readjusted. Optionally, the fiber 1726 includes a molded or cast coil portion 1730. The molded or cast coil 1730 takes up slack in the fiber 1736 when the jaw (not shown) is in a closed position.

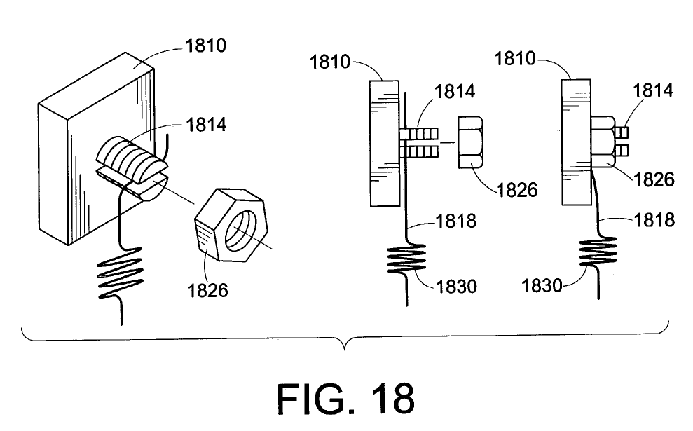

Referring to FIG. 18, a slotted nut and bolt bracket 1810 includes a slotted bolt 1814. In use, a restraining fiber 1818 is threaded through a slot 1822 in the slotted bolt and held in an appropriate position. A nut 1826 is then threaded onto the slotted bolt 1814 and tightened down onto the fiber 1818. The nut 1826 frictionally holds the fiber 1818 against the bracket 1810. Loosening the nut 1826 allows the position of the fiber to be readjusted. Optionally, the fiber 1818 includes a molded or cast coil portion 1830. The molded or cast coil 1830 takes up slack in the fiber 1818 when the jaw (not shown) is in a closed position.

Some patients may object to having their jaw movement permanently restrained for the entire weight loss program. For these patients, a semi-permanent jaw restraint may be sufficient.

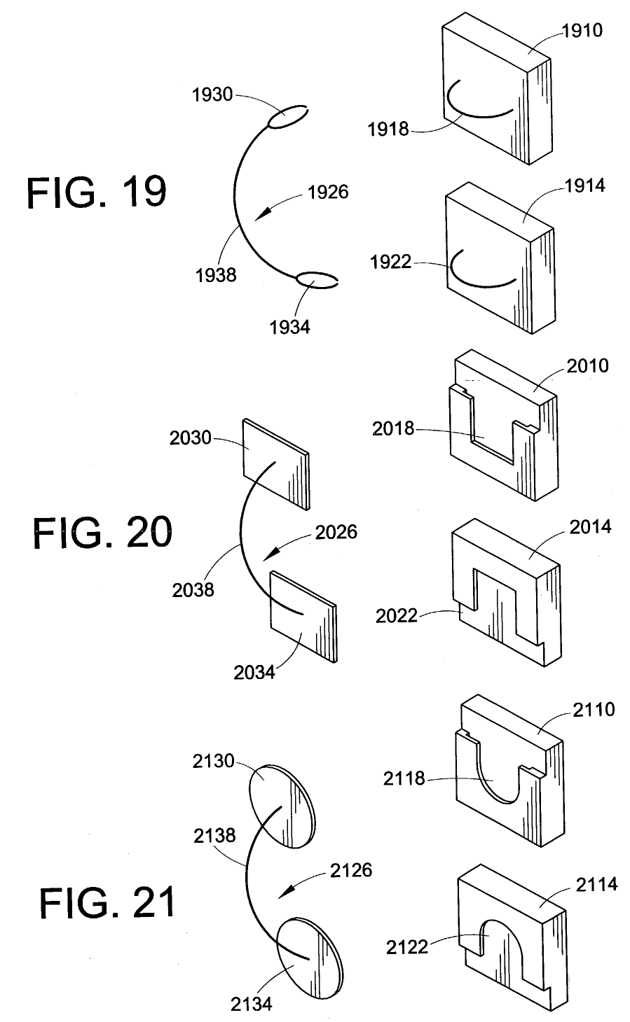

For example, referring to FIG. 19, a set of orthodontic brackets 1910, 1914 are mounted to upper and lower teeth as described above. The brackets 1910, 1914 include connector elements such as, for example, female portions or rings 1918, 1922. A temporary restraint element 1926 includes connectors such as, for example, releasable “lobster claw” clips 1930, 1934 for releaseably connecting the restraint element 1926 to the rings 1918, 1922. The clips 1930, 1934 are linked together by a restraint 1938. The restraint 1938 can be a chain, fiber, memory metal band, or similar to any of the other restraining elements described above. As described above in reference to the more permanent installations, symmetric sets of brackets 1910, 1914 are preferably installed on either side of the mouth. Restraint synchronization is carried out in a manner similar to that described above. For example, a bite block is sized or adjusted and the patient is asked to bite down on it. Where the restraint 1938 is a fiber, the fiber can then be cut to size and tied to the clips 1930, 1934. Where the restraint 1938 is a chain, the chain is sized by selecting the appropriate number of links and coupling the restraint 1938 to the clips 1930, 1934. Similar procedures can be followed with regard to memory metal bands and the other kinds of restraint elements such as those described above.

The clips 1930, 1934 allow the patient to connect and disconnect the restraint element 1926, thereby removing some of the inconvenience of the restraint when it isn't necessary and providing the useful function of the restraint when it is beneficial. For example, the patient can clip the restraint elements 1926 into the sets of brackets 1910, 1914 before each meal, or, for example, once each day. For example, the clips 1930, 1934 are snapped into the female portions or rings 1918, 1922 of the brackets 1910, 1914 and held in place by a latching mechanism (not shown).

Due to imperfections in the symmetry of the mouth and/or the symmetry of the installation of the brackets 1910, 1914 on either side of the mouth, it may be that the restraint element 1926 associated with each set of brackets 1910, 1914 is a different length. Therefore, since the restraints 1938 are removable, the patient should be aware of which restraint is associated with which set of brackets. For example, the restraints 1938 can be color coded or otherwise tagged. Alternatively, the patient should test restraint synchronization each time the restraint elements 1926 are installed. If one restraint 1926 appears to engage for one or more other restraint elements 1926, the patient should make another attempt to properly locate the restraint elements 1926.

Referring to FIG. 20, a second semi-permanent restraint system includes orthodontic brackets 2010, 2014, which are mounted to selected teeth of a patient by any of the means described above. The brackets 2010, 2014 include female portions or pockets 2018, 2022. Temporary restraint elements 2026 include male end pieces 2030, 2034 for being received in the pockets 2018, 2022, respectively. For example, the male end pieces 2030, 2034 are rectangular tabs. The rectangular tabs are linked together by a restraint 2038. For example, the restraint 2038 is similar to the restraint 1938 described in reference to FIG. 19 and is sized and installed accordingly. Similarly, symmetric sets of brackets 2010, 2022 and restraint elements 2026 are preferably installed on either side of the mouth. Before each meal, or, for example, once each day, the patient installs the restraint elements 2026 into the brackets 2010, 2014. For example, the male end pieces 2030, 2034 are snapped into the female portions 2018, 2022 of the brackets 2010, 2014 and held in place by a latching mechanism (not shown). For example, end caps can hold the male end pieces in the pockets 2018, 2022. Alternatively, camming style latches or other kinds of latching mechanisms can grasp the end pieces 2030, 2034. After the meal, or a bed time, the patient can remove the temporary restraining element 2026. For example, the patient can remove the end caps, overcome the forces of a latching mechanism or activate a release device. Of course, the cautionary comments made regarding restraint synchronization in reference to FIG. 19 apply to the embodiment described in reference to FIG. 20.

A third semi-permanent restraining system includes orthodontic brackets 2110, 2124 which include female portions or pockets 2118, 2122 and a temporary restraint element 2126. The temporary restraint element 2126 includes male end pieces 2130, 2134. The male end pieces 2130, 2134 are linked together by a restraint 2138. The restrains is similar to the restraints described in reference to FIG. 19 and FIG. 20. The shape of the male end pieces 2130, 2134 and the female portions or pockets 2118, 2114 are different than the shape of the male end pieces 2030, 2034 and female portions or pockets 2018, 2022. For example, the male end pieces 2130 and 2134 are in the form of circular tabs, and the female portions 2118, 2114 are shaped to receive them. However, in installation, operation, and most other respects, the third semi-permanent restraint system is the same as the system described in reference to FIG. 20.

Some patients may object to the bonding of brackets to their teeth. For those patients, removable trays can be employed to restraint jaw movement.

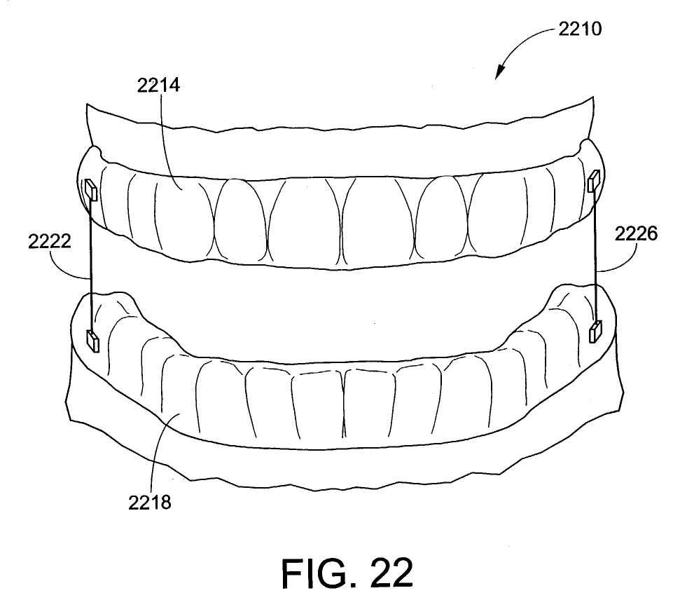

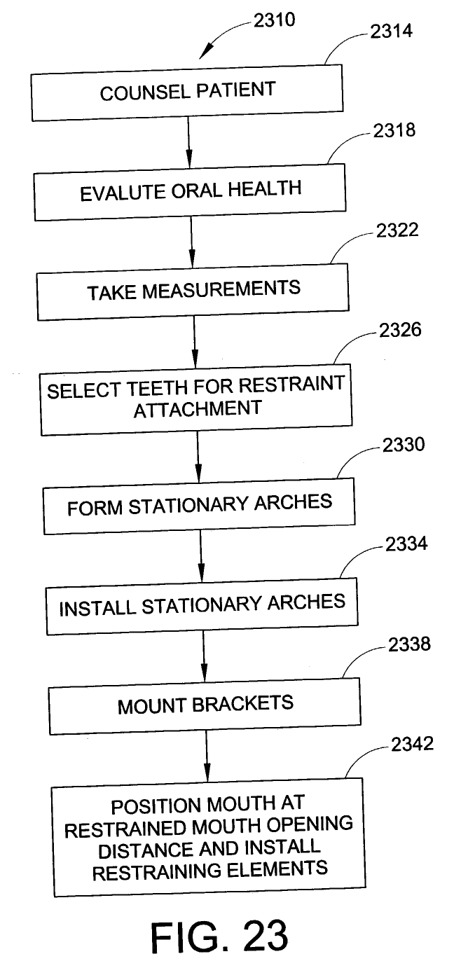

Referring to FIG. 22, an impression is made of the upper and lower teeth of the patient. The impression is used to make a tightly fitting custom set of trays 2210 for the patient. The trays snap over the teeth and are firmly held in place by the custom molded, interlocking nature of the trays with contours or undercuts of the teeth. An upper tray 2214 is linked to a lower tray 2218 by restraints 2222, 2226. Due to the perspective nature of FIG. 22, the restraints appear to be different lengths. However, the restraints 2222, 2226 are sized for synchronization so that the load of jaw restraining is shared as equally as possible between the two restraints 2222, 2226 and their associated trays and teeth. While the illustrated embodiment includes only a single upper tray 2214 and a single lower tray 2218, it is not always necessary to include the front or anterior teeth in the restraining system. In those cases, the anterior portion of the trays may be removed (or not included in the molding operation) leaving two upper trays and two lower trays for fitting over, for example, the left and right upper molars and the left and right lower molars, respectively. In any event, the upper and lower trays can be removed along with their linking restraints 2222, 2226 at the discretion of the patient. For example, the patient can install the trays before every meal or upon waking. The trays can be removed after every meal or, for example, before going to sleep.

Magnets can be associated with the brackets 1910, 1914, 2010, 2014, 2110, 2114 in manners and for purposes similar to those described above in reference to the more permanent type installations. Furthermore, similar magnets can be associated with the trays 2210, 2214.

Additionally, if the restraints are iron 1938, 2038, 2138, 2238 based, for example, made of stainless steal, or other wise magnetic, magnets can be included in association with the brackets, restraints or trays to help coil up the restraints when the mouth of the patient is closed. For example, the clips 1930, 1934, or male tabs 2030, 2034, 2130, and 2134 can comprise magnets or magnets can be included in the brackets 1910, 1914, 2010, 2014, 2110, 2114. Coiling magnets can also be molded into the trays 2210, 2214.

The brackets 1910, 1914, 2010, 2014, 2110, 2114 and trays 2210, 2214 can also be used in conjunction with stationary arches.

While the invention has been described, up until now, in terms of direct bonding of fibers and the use of brackets bonded to teeth, other mounting apparatus can be used. For example, interdental brackets that involve a mounting pin or wire inserted through an interdental space between teeth can also be used. A nut and a bracket mounted on the pin sandwich portions of teeth on either side of the interdental space. The nut and bracket act as clamping elements. The bracket can be any bracket, such as, for example, modified versions of the brackets described above in reference to FIG. 10-FIG. 22. Alternatively, brackets can be mounted with orthodontic bands. The bands are wrapped around the entire circumference of the tooth. The band replaces the bracket pad and adhesive. Any of the bracket mechanism, including those described above, in reference to FIG. 10-FIG. 22 can be attached to the band.

In summary, a method 2310 operative to help a patient lose weight should include a consultation 2314. In the consultation, the patient is told about the method and apparatus. The various apparatus options are described. The purpose of the apparatus is also explained and the patient's level of motivation is evaluated. If the patient indicates a desire to use the apparatus as an aid in losing weight, the oral health of the patient is evaluated 2318.

The oral health evaluation includes 2318, for example, a standard medical/dental status review and documentation procedure. The evaluation involves a blood work up, vital signs (height, weight, blood pressure, temperature), and a general dental evaluation of the teeth and gums. The oral health evaluation 2318 facilitates a treatment plan design. For example, the oral health evaluation aids in the selection of the best teeth to receive the appliance. The oral heath evaluation 2318 also uncovers any contraindications that may be associated with the patient.

Contraindications to the use of the appliance may include, for example, a lack of posterior teeth, a lack of any teeth at all, rotted or decayed teeth, and severe gum disease.

If no contraindications are noted in the oral health evaluation 2318, measurements are taken 2322 related to the mouth of the patient. For example, a centric relation and a maximum speech distance measurement are taken. The centric relation is a measurement of the relation of the teeth when the mandibular condyle is in the most anterior superior position. The maximum speech distance measurement is the maximum distance a patient needs be able to move the jaw in order to complete every sound needed during speech. This distance may be a practical limit on an amount of jaw movement restriction that can be provided to the patient. These measurements and a consideration of the patients eating habits and life style are taken into consideration when selecting a restrained distance.

The oral health evaluation 2318 and measurement collection 2322 can also be helpful in selecting teeth 2326 or positions within the mouth for attaching restraining fibers or mounting restraining brackets. Preferably, upper and lower first and second molars are selected. However, if one or more of those teeth are unavailable or contraindicated, bicuspids can also be used. Selecting more anterior teeth has some drawbacks. For example, the anterior teeth have a reduced root surface area. This reduced surface area leads to a reduction in tooth anchorage and an increased risk of tooth loosening and movement. Selecting more anterior teeth also has cosmetic drawbacks in that others can more easily see the appliance.

Where indicated, or where extra precautions are desired, stationary arches or arch segments are designed and formed 2330. Preferably, stationary arches are formed by first taking impressions of the upper and lower jaws. The impressions are sent to an orthodontic lab. The lab fabricates lingual holding arches. This procedure may be initially bypassed, and only used in cases where unwanted tooth movement is noted. When used, the arches are, of course, installed 2334. Preferably, the arches or arch segments are installed on the lingual side of the teeth.

Restraining fibers or brackets are then mounted or bonded to the selected teeth. Where brackets are used restraining fibers or elements are installed in the brackets. In order to install the restraining fibers or elements, the patient's mouth is positioned at a selected restricted maximal opening. Preferably, the opening is not less that the maximum speech opening distance. However, preferably the restricted maximal opening distance is otherwise as small as can be tolerated by the patient. The patient's mouth is held at the restricted maximal opening by placing an adjustable bite block into the patient's mouth. The bite block is selected or adjusted to correspond to the selected restricted maximal opening. The patient is asked to bite down on the block, and the restraining fibers are installed and synchronized either by direct bonding or through the use of brackets that facilitate fiber adjustment and synchronization such as those described in reference to FIG. 10-FIG. 22.

The invention has been described with reference to particular embodiments. Modifications and alterations will occur to others upon reading and understanding this specification. It is intended that all such modifications and alterations are included insofar as they come within the scope of the appended claims or equivalents thereof.

What is claimed is:

1. An appliance kit for installation in a mouth of a patient, the installed kit operative to limit an extent to which the mouth can be opened, thereby slowing the ingestion of food, reducing the volume of food ingested before a the patient feels satiated and helping the patient to lose weight, the appliance kit comprising:
a restraining device made from material selected from the group comprising of orthodontic wire, fiber, mesh, plastic, metal, memory metal and ribbon, the restraining device operative to allow the mouth to be opened enough to speak and for other purposes, while reducing an amount of food that the patient can chew in any one bite;
a means for attaching the restraining device between a portion of an upper jaw and a portion of a lower jaw of the patient
an upper stationary arch; and,
a lower stationary arch, the upper and lower stationary arches to be attached to portions of the upper and lower jaw respectively, and to respective upper and lower teeth, the upper and lower teeth selected for attaching the restraining device to, the arches operative to counter act forces tending to withdraw the selected upper and lower teeth.

2. The appliance kit of claim 1 wherein the attachment means comprises:
an upper bracket to be attached to a portion of the upper jaw and a lower bracket to be attached to a portion of the lower jaw, the brackets each providing an anchor point for attaching the restraining device.

3. The appliance kit of claim 1 wherein the attachment means comprises:
an upper bracket to be bonded to a tooth of the upper jaw and a lower bracket to be bonded to a tooth of the lower jaw, the brackets each providing an anchor point for attaching the restraining device.

4. The appliance kit of claim 1 wherein the attachment means comprises:
an adhesive operative to bond the restraining device directly to a tooth of the upper jaw and a tooth of the lower jaw

5. The appliance kit of claim 1 wherein the restraint further comprises:
a predetermined length of the selected material, the predetermined length being based on measurements made of the patients mouth.

6. The appliance kit of claim 1 wherein the restraint further comprises:
a plurality of strips of the selected material

7. The appliance kit of claim 1 further comprising:
a magnetic restraint including an upper magnet and a lower magnet, the upper and lower magnets operative to be mounted to portions of the upper and lower jaw respectively, the magnets to be mounted with dissimilar magnetic poles facing each other, whereby a magnetic interaction between the magnets opposes the opening of the mouth, thereby slowing the chewing process.

8. The appliance kit of claim 1 further comprising:
a bite block operative to set a jaw opening distance while measurements are taken and while the restraining device is installed.

9. The appliance kit of claim 2 wherein the upper bracket and the lower bracket each include one of a female and a male connection element and the restraining device includes two male or female connection elements for removably connecting to the female or male connection element of the upper and lower brackets.

10. The appliance kit of claim 9 further including a magnet in association with at least one of the upper bracket and the lower bracket and wherein the restraining device includes a chain and the magnet is operative to assist in the coiling of the chain when the jaw is closed.

11. The appliance kit of claim 1 where in the means for attaching the restraining device between a portion of the upper jaw and the lower jaw comprises and upper tray and a lower tray made to conform to teeth of the upper jaw and the lower jaw respectively whereby the trays can be snapped in place over their respective teeth and removed from the teeth by the patient.

12. The appliance kit of claim 11 further including a magnet in association with at least one of the upper tray and the lower tray and where in the restraining device includes a chain and the magnet is operative to assist in the coiling of the chain when the jaw is closed.

13. A method for helping a patient lose weight; the method comprising the steps of:
selecting an upper tooth from an upper jaw of a mouth of the patient, for attaching a restraining device;
selecting a lower tooth from a lower jaw of the mouth of the patient, for attaching the restraining deceiving to, the selected upper and lower teeth positioned in opposed relation within the mouth of the patient.
installing an upper stationary arch by attaching the upper stationary arch to the selected upper tooth and attaching the upper stationary to at least one other portion of the upper jaw; and
installing a lower stationary arch by attaching the lower stationary arch to a tooth selected to be an attachment point for the restraining device and attaching the lower stationary at least one other portion of the lower jaw.
determining a restrained mouth opening distance; and
installing a mouth opening restraining device so as to restrain the mouth from being opened beyond the determined restrained mouth opening distance.

14. The method of claim 9 wherein the step of installing a mouth opening restraining device comprises:
cutting a ribbon of dental reinforcing ribbon to a predetermined length based on the determined restrained mouth opening distance;
bonding the length of dental reinforcing ribbon to an upper tooth and to a lower tooth.

15. The method of claim 9 wherein the step of installing a mouth opening restraining device comprises:
bonding a length of dental reinforcing ribbon to an upper tooth and to a lower tooth.

16. The method of claim 9 wherein the step of installing a mouth opening restraining device comprises:
attaching an upper bracket to a selected tooth of the upper jaw;
attaching a lower bracket to a tooth of the lower jaw, the lower tooth positioned opposite the selected tooth of the upper jaw; and,
attaching a restraining device to the upper and lower brackets.

17. The method of claim 9 further comprising, before the step of installing a mouth opening restraining device, the steps of:
selecting a bite block based on the determined restrained mouth opening distance; and,
biting on the bite block to hold the mouth open a distance related to the determined restrained mouth opening distance.

18. An appliance kit for installation in a mouth of a patient, the installed kit operative to limit an extent to which the mouth can be opened, thereby slowing the ingestion of food, reducing the volume of food ingested before a the patient feels satiated and helping the patient to lose weight, the appliance kit comprising:
a restraining device made of a plurality of pieces of orthodontic reinforcing ribbon, the restraining device operative to allow the mouth to be opened enough to speak and for other purposes, while reducing an amount of food that the patient can chew in any one bite; and
a bonding agent operative to bond the plurality of pieces of orthodontic reinforcing ribbon between respective teeth of the maxilla and mandible of the mouth of the patient.

19. The appliance kit of claim 14 further comprising:
an upper stationary arch; and,
a lower stationary arch, the upper and lower stationary arches operative to be attached to a plurality of teeth of the of the maxilla and mandible respectively including the respective teeth, the stationary arches operative to counteract forces applied to the respective teeth by the restraining device, thereby preventing the respective teeth from being loosened.

20. The appliance kit of claim 14 further comprising:
at least one pair of magnets operative to be mounted to opposed upper and lower teeth and oriented with opposite magnetic poles adjacent each other, thereby providing an attractive force that must be overcome by the patient during the beginning of chew, thereby slowing the chewing process.

21. An appliance kit for installation in a mouth of a patient, the installed kit operative to limit an extent to which the mouth can be opened, thereby slowing the ingestion of food, reducing the volume of food ingested before a the patient feels satiated and helping the patient to lose weight, the appliance kit comprising:
at least two sets of upper and lower brackets operative to be mounted on symmetrically opposed upper and lower teeth on the right and left side of the mouth of the patient;
a set of at least two restraining elements, the restraining elements co-operating with the brackets to provide a means for synchronizing the activation of the fibers when the fibers and brackets are mounted to teeth of the patient to limit the opening of the patients mouth.

22. The appliance kit of claim 21 wherein the brackets and the restraining elements each include mating connectors for selectively linking the upper brackets to respective opposing brackets by selectively connecting and disconnecting the restraining elements to respective upper and lower brackets.

23. The appliance kit of claim 22 further including at least one a magnet in association with at least one of the upper brackets and the lower brackets and where in the restraining device includes a chain and the magnet is operative to assist in the coiling of the chain when the jaw is closed.

24. An appliance kit for installation in a mouth of a patient, the installed kit operative to limit an extent to which the mouth can be opened, thereby slowing the ingestion of food, reducing the volume of food ingested before a the patient feels satiated and helping the patient to lose weight, the appliance kit comprising:
a set of at least two trays operative to be mounted on opposed upper and lower teeth of the patient;
a set of at least two restraint elements attached to the at least two trays, the restraint elements sized to be activated to restrain a movement of the mouth of the patient in a synchronized manner when the trays are mounted to teeth of the patient and the mouth of the patent is opened.

25. The appliance kit of claim 24 further including a magnet in association with at least one of the upper tray and the lower tray and where in the at least two restraining devices include at least two chains and the magnet is operative to assist in the coiling of the chain when the jaw is closed.
